(19) United States
(12) Patent Application Publication
(10) Pub. No.: US 2009/0099519 A1
(43) Pub. Date: Apr. 16, 2009

(54) ADVANCED ABDOMINAL DRESSING FOR THE TREATMENT OF THE POSTOPERATIVE HYPOTHERMIC PATIENTS WITH AN OPEN ABDOMEN

(75) Inventor: Mark Kaplan, Huntington Valley, PA (US)

Correspondence Address: VOLPE AND KOENIG, P.C., UNITED PLAZA, SUITE 1600, 30 SOUTH 17TH STREET, PHILADELPHIA, PA, 19103, US

(73) Assignee: ALBERT EINSTEIN HEALTHCARE NETWORK, Philadelphia, PA (US)

(21) Appl. No.: 12/206,447

(22) Filed: Sep. 8, 2008

Related U.S. Application Data
(60) Provisional application No. 60/967,881, filed on Sep. 7, 2007.

(51) Int. Cl. A61F 7/12; A61M 1/00; A61B 19/08

(52) U.S. Cl. 604/113; 604/313; 128/853

(57) ABSTRACT

An apparatus for abdominal dressing that adds liquid coils in the form of a manifold is disclosed. The manifold includes heating coils to warm the bowel with heated water in a counter current method. This takes advantage of the large surface area of the bowel to exchange heat with the heater coils, but allows the abdominal dressing to maintain the initial function: remove pathologic fluid from the abdomen. Methods of treating patients with an open abdomen are also disclosed.

### CROSS REFERENCE TO RELATED APPLICATIONS

This application claims the benefit of U.S. provisional application No. 60/967,881, which was filed Sep. 7, 2007 and is incorporated herein by reference as if fully set forth.

### FIELD OF INVENTION

This application relates to vacuum assisted closures and prevention of hypothermia in trauma patients, including post-operative patients. The present invention also relates to the healing of wounds.

### BACKGROUND

Wound closure involves the inward migration of epithelial and subcutaneous tissue adjacent the wound. This migration is ordinarily assisted through the inflammatory process, whereby blood flow is increased and various functional cell types are activated. Through the inflammatory process, blood flow through damaged or broken vessels is stopped by capillary level occlusion, whereafter cleanup and rebuilding operations may begin. Unfortunately, this process is hampered when a wound is large or has become infected. In such wounds, a zone of stasis may develop. This zone is an area of localized tissue near the surface of the wound that swells and restricts the flow of blood to the tissues.

Without sufficient blood flow, the epithelial and subcutaneous tissues surrounding the wound not only receive diminished oxygen and nutrients, but are also less able to successfully fight bacterial infection and are less able to naturally close the wound. Until recently, such difficult wounds were addressed through the use of mechanical closure techniques that utilized devices such as sutures or staples.

Although still widely practiced and often effective, mechanical closure techniques suffer a disadvantage in that they produce tension on the skin tissue adjacent the wound. In particular, the tensile force required in order to achieve closure using sutures or staples causes very high localized stresses at the suture or staple insertion point. These stresses commonly result in the rupture of the tissue at the insertion points, which can eventually cause wound dehiscence and additional tissue loss.

Additionally, some wounds harden and inflame to such a degree due to infection that closure by stapling or suturing is not feasible. Wounds not reparable by suturing or stapling generally require prolonged hospitalization, with its attendant high cost, and major surgical procedures, such as grafts of surrounding tissues. Examples of wounds not readily treatable with staples or suturing include large, deep, open wounds; decubitus ulcers; ulcers resulting from chronic osteomyelitis; and partial thickness burns that subsequently develop into full thickness burns.

Wounds can also be treated with vacuum assisted therapy. Vacuum assisted therapy can be accomplished with apparatuses that drain wounds by applying negative pressure to the wound. When applied over a sufficient area of the wound, such negative pressures have been found to promote the migration toward the wound of epithelial and subcutaneous tissues. The application of negative gauge pressure to a wound typically involves mechanical-like contraction of the wound with simultaneous removal of excess fluid. In this manner, vacuum assisted therapy augments the body's natural inflammatory process while alleviating intrinsic side effects. In particular, vacuum assisted therapy can alleviate edema caused by increased blood flow absent the necessary vascular structure for proper venous return. As a result, vacuum assisted therapy has been successful in the promotion of wound closure, healing many wounds previously thought largely untreatable.

In patients subjected to surgery involving the abdomen, the use of the open abdomen for post-operative management has increased significantly in the past decade. Further, vacuum assisted therapy has been an effective management tool for this complex class of patients. The major goal in managing post-operative abdomen surgery patients is to prevent the development of increased pathologic pressures in the abdomen, which have life threatening consequences if not treated. Another major concern in patients with trauma, including trauma such as an open abdomen, is the loss of heat. In the case of the open abdomen, the exposed bowel and fluid losses may lead to a potentially low body temperature state called hypothermia. This is the second leading cause of death in trauma patients. To date there are no efficient therapies to treat trauma or postoperative hypothermia.

### SUMMARY

In a first aspect, the invention relates to a temperature control vacuum assisted therapy device for providing therapy to a patient. The device includes a dressing that includes a cover layer, an encapsulated pad, a drape, and a pressure adapter. The cover layer is fenestrated and adapted to be placed within a wound. The encapsulated pad protrudes at least partially through the fenestration of the cover layer and is adapted to allow negative pressure to be applied to the wound. The drape covers the cover layer and encapsulated pad and is capable of providing a seal between the patient and the drape, the drape includes an opening. The pressure adapter is mounted above the opening of the drape and includes fittings that allow negative pressure to be applied to the wound through the pressure adapter. The device also includes a temperature control operatively connected to the dressing. The temperature control include a thermal element that is closely associated with the cover layer and adapted to be placed within the wound.

In a second aspect, the invention relates to a method of preventing or treating hypothermia in a post-operative abdominal surgery patient that has an open abdomen. The method includes providing a device having a dressing that includes a cover layer, an encapsulated pad, a drape, and a pressure adapter. The cover layer is fenestrated and is adapted to be placed within a wound. The encapsulated pad protrudes at least partially through the fenestration of the cover layer and is adapted to allow negative pressure to be applied to the wound. The drape covers the cover layer and the encapsulated pad and is capable of providing a seal between the patient and the drape. The drape also includes an opening. The pressure adapter is mounted above the opening of the drape and includes fittings that allow negative pressure to be applied to the wound through the pressure adapter. The provided device also has a temperature control operatively connected to the dressing. The temperature control includes a thermal element that is closely associated with the cover layer and is adapted to be placed within the open abdomen. The method also includes applying the device to the open abdomen with the thermal element within the open abdomen and providing heat to the patient through the thermal element.

### BRIEF DESCRIPTION OF THE DRAWINGS

The following detailed description of the preferred embodiment of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1 provides an exploded view an embodiment of the invention with a dressing that can be used to provide negative pressure on a wound and provide or remove heat with a temperature control.

FIG. 2 illustrates an embodiment of a dressing that includes temperature control thermal element coils in the form of a manifold.

### DETAILED DESCRIPTION OF THE PREFERRED EMBODIMENTS

Certain terminology is used in the following description for convenience only and is not limiting. The words “right,” “left,” “top,” and “bottom” designate directions in the drawings to which reference is made.

As used herein, “wound” refers to any wound including accidental wounds or wounds received as the result of surgery. Wounds as the result of surgery may include wounds that have not healed or wounds purposefully left open such as a post-operative open abdomen.

As used herein, “patient” refers to a human or non-human subject in need of wound therapy, warming, or cooling.

The words “a” and “one,” as used in the claims and in the corresponding portions of the specification, are defined as including one or more of the referenced item unless specifically stated otherwise. This terminology includes the words above specifically mentioned, derivatives thereof, and words of similar import.

In an embodiment of the invention, a device for vacuum assisted therapy includes a negative pressure dressing that can be used to apply negative pressure to a wound. Further, the device includes a temperature control with an internal thermal element operatively connected to the negative pressure dressing that can be used to provide heat to a patient or remove heat from the patient. In a preferred embodiment, the thermal element is closely associated or part of portions of the dressing that contact the contents of the patient's wound. In a further preferred embodiment, the thermal element is a heating element. In another preferred embodiment, the thermal element is a cooling element. In other embodiments, the invention includes methods of controlling wound closure by applying the device to a wound and providing or removing heat. In still further embodiments, the invention includes methods to prevent hypothermia in trauma patients by applying the device to a wound and providing heat to the patient with the temperature control.

The Vacuum Assisted Closure (or “V.A.C.®”) system by Kinetic Concepts, Inc. (“KCI”) of San Antonio, Tex. is an example of an apparatus for vacuum assisted therapy of open wounds. The V.A.C.® system and other abdominal wound treatment systems are described in “Kaplan M., Banwell P., Orgill D., Ivatury R., Demitriades D., Moore F., Miller P., Nicholes J., Henry S., Guidelines for the Management of the Open Abdomen, Supplement to Wounds: A Compendium of Clinical Research and Practice, October 2005., which is incorporated herein by reference as if fully set forth. Vacuum assisted therapy has also been described in U.S. Pat. Nos. 4,969,880; 5,100,396; 5,261,893; 5,527,293; 6,071,267; 5,636,643; 5,645,081; 6,117,111; 6,142,982; 6,345,623; 6,553,998; 6,814,079; and U.S. pre-grant publication No. 2007/0073200; all of which are incorporated herein by reference as if fully set forth. Embodiments of the present invention include modification of any of the vacuum assisted therapy device described in these references with an internal thermal element.

In an embodiment, the V.A.C.® abdominal dressing developed by KCI may be modified with temperature control. The original dressing was designed to maintain the integrity of the abdominal viscera and to facilitate the closure of the open abdomen. However, hypothermia is a significant problem in patients with an open abdomen and is not treated with current vacuum assisted therapy devices. Instead, hypothermia is potentially exacerbated when vacuum assisted therapy devices are applied to an open abdomen. Additionally, addition of heat exterior to the wound, such as described in U.S. pre-grant publication No. 2007/0073200, is insufficient to overcome patient cooling due to vacuum assisted therapy. In some cases, the constituents of the vacuum assisted therapy devices act as thermal insulators and impede transfer of heat by insulating the patient or the wound from a thermal element. For example, the V.A.C.® abdominal dressing includes an foam pad that distributes vacuum and allows for efficient withdrawal of fluid. However, the foam pad would insulate the contents of the abdomen from heat applied above the pad.

Combining vacuum assisted therapy with flushing an open abdomen with liquid of a desired temperature may result in undesirable fluid or electrolyte exchange. Embodiments of the present invention allow for heating of the open abdomen using a counter current technique that does not require exchange of fluids and will not effect the overall function of the dressing. Because the overall function of the dressing remains, the viscera can be maintained and drainage of accumulated abdominal fluid, which is secondary to the acute process, can be achieved. The temperature control modified dressing can be modified to fit the patient, similar to the fit achieved with the original dressing. This is unique because other forms of warming, including heating lamps, warming blankets; warmed fluids and blood are not effective over a short but critical time period. In this embodiment, the device will warm the large exposed surface area of the bowel to exchange heat and rapidly warm the patient. A primary use of the device could be in trauma patients with severe injuries that require open abdomens since these patients have the highest risk of death and bleeding caused by hypothermia. The device could also be used in the hypothermic patient that was cooled as a result of exposure where the patient requires rapid warming. Warming a hypothermic patient with embodiments of the present invention could be used as an alternative to cardiopulmonary bypass, which has significant risks and associated morbidity. In preferred embodiments, methods of preventing hypothermia are carried out by placing the temperature control modified vacuum assisted therapy device on a post-operative open abdomen and providing heat to the patient's bowels.

In alternate embodiments, a temperature control modified vacuum assisted therapy device could also be used for cooling. In these embodiments, the patient could be a feverish patient or a patient in need of metabolic slowing. In either heating or cooling embodiments, the internal thermal element can be a liquid manifold of the device, which can be connected to external tubing to provide heating or cooling liquid within the manifold. The liquid can be heated by a counter current heater.

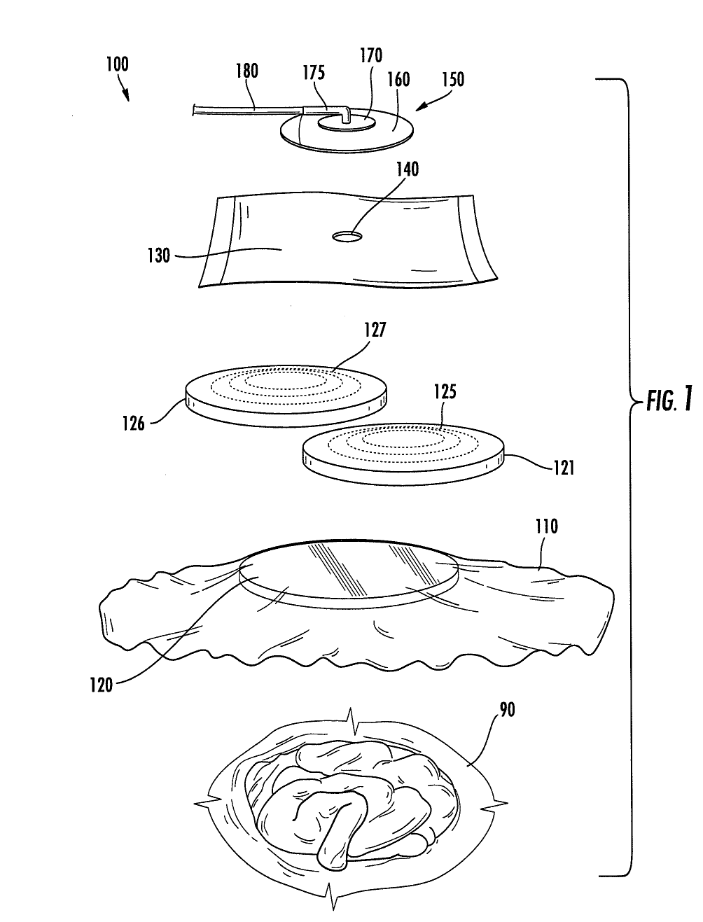

Referring to FIG. 1, an embodiment of the present invention includes a dressing 100 that can be used to apply negative pressure and heating or cooling temperatures to a wound 90. Heating or cooling temperatures are provided by temperature control 105, which is operably connected to the dressing 100 such that heat can be transferred between the temperature control and the wound 90.

In a preferred embodiment, the wound 90 is an open abdomen. FIG. 1 also illustrates a cover layer 110 that contacts the wound 90. Cover layer 110 is a fenestrated layer that includes an opening through which at least a portion of encapsulated pad 120 protrudes into wound 90. In this embodiment, fluid can pass from the wound 90 and through the pad 120. In an alternate embodiment, encapsulated pad 120 is positioned on the wound 90 and over cover layer 110. In this alternative, openings or pores in cover layer 110 allow passage of fluid between wound 90 and the dressing 100. In further alternate embodiments, cover layer 110 overlays encapsulated pad 120 but openings or pores within cover layer 110 allow passage of fluid from the wound through pad 120 and then through cover layer 110. In still further alternate embodiments, the encapsulated pad 120 is absent and openings or pores in cover layer 110 allow passage of fluid. Embodiments with encapsulated pad 120 are, however, preferred since the pad 120 facilities placement of the dressing 100 in the wound 90 and minimizes dressing shift.

Encapsulated pad 120 is preferably an open face foam pad. In preferred embodiments, the encapsulated pad 120 is positioned to deliver a constant negative pressure to the wound that causes a macro deformational strain and contacts the wound.

In preferred embodiments, the dressing 100 is placed in an open abdomen and the cover layer 110 is placed under the facia. In these embodiments, cover layer 110 can be placed or wrapped over one or more of the contents of the bowel, including exposed organs, the greater omentum, the lesser omentum, exposed intestines, or other abdominal contents. Also in these embodiments, a temperature control 105 with a conformable thermal element is provided. The conformable thermal element is positioned near to or integral to cover layer 110 and warmth can be provided in proximity to the contents of the bowel. In a preferred embodiment, the thermal element is embedded in cover layer 110. In another preferred embodiment, the cover layer 110 is non-adherent. In a still more preferred embodiment, the dressing provides a closed system where cover layer 110 is made of material that does not allow intraperitoneal fluid or electrolyte exchange. An example of such a material is polyurethane.

Still referring to FIG. 1, embodiments are illustrated with secondary pads 121 and 126. The optional secondary pad 121 can be positioned above encapsulated pad 120. One or more optional secondary pads such as pad 126 can also be positioned above optional secondary pad 121. Secondary pads can be added to adapt the dressing to the wound 90 shape and size. Preferably, at least one of the pads 120, 121, or 126, when used alone or in combination, is made of foam. As illustrated in FIG. 1, in still further embodiments, secondary pads can include perforations 125, 127 and the perforations 125, 127 allow the pads to be further adapted to the shape and size of the wound 90.

In embodiments were pad 120 foam contacts a wound healing surface, the foam preferably has relatively few open cells in contact with the areas upon which cell growth is to be encouraged so as to avoid unwanted adhesions. In these embodiments, the pad 120 has a sufficient number of open cells so that drainage and negative pressure therapy may continue unimpaired.

Referring to FIG. 1, a drape 130 overlays the wound 90, cover layer 110, encapsulated pad 120, and, if present, secondary pads 121, 126. In an embodiment, more than one sheet of drape material is overlapped or otherwise connected to form drape 130 and cover the wound 90. In another embodiment, more than one sheet of drape material is stacked one upon another to form a thicker drape 130. In a preferred embodiment, four sheets of drape material are stacked one upon another to form drape 130. As shown in the embodiment illustrated in FIG. 1, the drape 130 includes an opening 140 through which negative pressure can be applied.

Still referring to FIG. 1, a pressure adapter 150 is mounted over opening 140. The pressure adapter 150 includes fittings that allow negative pressure to be applied through the pressure adapter. In the preferred embodiment illustrated in FIG. 1, a sealing ring 160 surrounds the opening 140 and contacts the drape 130. A connector 170 contacts sealing ring 160 and provides a narrowed passage fitting 175, upon which a tube 180 can be mounted. Drape 130 and pressure adapter 150 provide closure to the dressing such that negative pressure drawn through tube 180 can be applied through the dressing and to the wound 90.

To provide negative pressure, a pump can be operably connected to the pressure adapter fittings 175 through tube 180. Further, a liquid collection flask may be interposed between the pump and the dressing along the path of the tube.

Alternate embodiments include placing the pad 120 in fluid communication with a vacuum source for promotion of fluid drainage by methods known in the art. In one alternate embodiment, the fluid communication may be established by connecting a tube to a dressing, such as that described in U.S. Pat. No. 6,345,623, corresponding to international application WO 99/13793, entitled “Surgical Drape and Suction Heads for Wound Treatment,” both of which are incorporated herein by reference as if fully set forth.

The temperature control 105 includes a thermal element that can provide heating or cooling temperatures. When the thermal element provides heat it is a heating element and when it provides cool temperatures it is a cooling element. The thermal element can be a liquid manifold. The thermal element can provide heat derived from chemical, electrical, or physical sources. Similarly, the thermal element can provide cooling, or withdraw heat, from chemical, electrical, or physical sources. In a preferred embodiment, a temperature control 105 includes a regulator that cools or heats a liquid that is circulated through a coil or manifold, which serves as the thermal element. The liquid can be delivered to the coils via a pump and tubing that is attached to the inlet and outlet channels of a coil. In another embodiment a heating or cooling gas may be circulated in a coil or manifold. In still another embodiment, the coil may be made of a conductive substance and heated or cooled by direct chemical, physical, or electrical devices.

The temperature control 105 is operably connected to the dressing 100 such that heat can be transferred between the patient and the temperature control 105. In preferred embodiments, the thermal element is placed within the wound or open abdomen; i.e., the thermal element is internal. Preferably, the thermal element is closely associated with the cover layer 110. In one embodiment, the thermal element is closely associated with the cover layer by being embedded in the cover layer 110. In an alternate embodiment, the cover layer is closely associated with the cover layer by constituting part of the cover layer 110; i.e., being integral to the cover layer 110. In still further embodiments, the thermal element is closely associated with the cover layer 110 by being reversibly or irreversible fixed or adhered thereto. In alternative embodiments, the thermal element may be interposed between the wound 90 and encapsulated pad 120.

In embodiments where liquid is circulated through the thermal element, the liquid can be circulated in a countercurrent fashion. In an abdominal dressing embodiment, a manifold includes the at least one coil to warm the bowel with heated liquid, preferably water, in a counter current method. In still more preferred embodiments, the heater coils that warm the bowel are closely associated with the cover layer 110 and/or the encapsulated pad 120. Further, cover layer 110 and/or encapsulated pad 120 are in close proximity to contents of the exposed bowel. Heating the open post-operative or trauma abdomen takes advantage of the large surface area of the bowel to exchange heat with the heater coils, but allows an abdominal dressing to maintain an initial function: removing pathologic fluid from the abdomen. The combination of vacuum assisted therapy and heating of the bowel thus provides a treatment affect for post-operative or trauma patients that is not available with simple vacuum assisted therapy. Nor is the affect available by adding a prior art heating method such as blankets or other sources of external heat to simple vacuum assisted therapy.

The components of a temperature control modified vacuum assisted therapy apparatus can be adhered to one another reversibly or irreversibly. In one embodiment, components are adhered to one another by the force of applied negative pressure. In another embodiment, adhesive holds one component to another. In another embodiment, at least one of the components is integral to one of the other components. In still other embodiments, components can be slidably connected, such as narrowed passage 175 and tube 180.

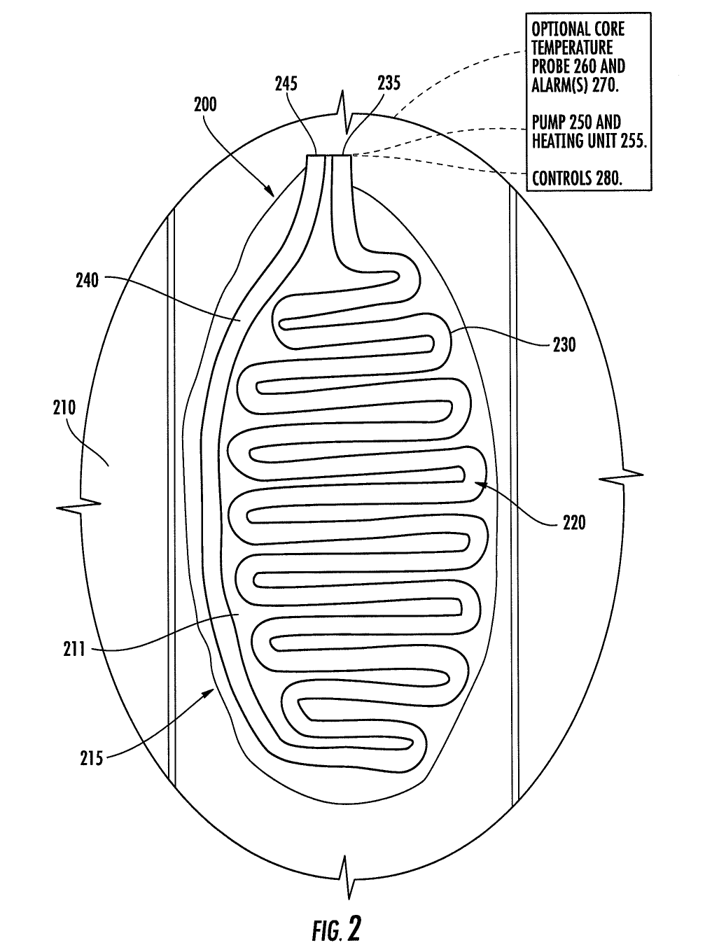

Referring to FIG. 2, an embodiment is illustrated that includes an abdominal dressing 200 with a heater coil depicted as coils 230, 240. The coils 230, 240 are in the form of a manifold 220. As illustrated, a patient's open abdomen 210 is fitted with the dressing 200. An encapsulated pad 211 fills the abdominal opening 215. However, as presented in FIG. 2, the pad is illustrated transparently and an internal manifold 220 is seen through the encapsulated pad 211. The dressing 200 includes a liquid inflow channel 235 and a liquid outflow channel 245. In a still more preferred embodiment, a pump mechanism 250 circulates liquid through the heater coil via channels 235, 245. Further, a regulator in the form of heater mechanism 255 is utilized to warm the circulating liquid. A core temperature probe 260 can be connected to a central temperature monitor, which can be used to monitor the patient's temperature or the dressing temperature. In a still further embodiment, an alarm 270 is operatively connected to the dressing 200 and can be set to alert the operator. The alarm 270 can be set to trigger the alert if the temperature of the patient or the dressing deviates from a pre-defined range. The alarm can also be set to trigger the alert if the temperature of the patient reaches a pre-determined extreme. In still further embodiments, the dressing 200 includes controls 280, which can be used to adjust liquid or heat flow through the manifold. The entire dressing can be used as a unit and function in the post operative period or in patients with hypothermia that need rapid warming.

In a preferred embodiment, a temperature control modified vacuum assisted therapy device can be used in combination with other devices that are used to address the problem of cooling or hypothermia. Other devices include, but are not limited to, blood and fluid warmers, warming blankets, and warm fluid irrigation. As previously noted, the present embodiments of a temperature control modified abdominal dressing include a thermal element that can be in thermal contact with a large surface area and large blood supply within a patient's open abdomen. The thermal transfer possible with the present embodiments is greater than that achieved with heating pads applied to the exterior of a wound. Also, the additional heat applied to the intestines may enhance blood flow, which can be compromised in a trauma or septic patient. A temperature control vacuum assisted therapy device may also improve blood coagulation, decrease the need for blood and blood products, and enhance the bodies' defenses against infection with the potential to lower complications and length of stay in the hospital.

All references cited are incorporated herein by reference as if fully set forth.

It is understood, therefore, that the invention is not limited to the particular embodiments disclosed, but is intended to cover all modifications which are within the spirit and scope of the invention as defined by the appended claims; the above description; and/or shown in the attached drawings.

What is claimed is:

1. A temperature control vacuum assisted therapy device for providing therapy to a patient, the device comprising:
(a) a dressing that includes a cover layer, an encapsulated pad, a drape, and a pressure adapter,
the cover layer is fenestrated and adapted to be placed within a wound,
the encapsulated pad protrudes at least partially through the fenestration of the cover layer and is adapted to allow negative pressure to be applied to the wound,
the drape covers the cover layer and the encapsulated pad, the drape is capable of providing a seal between the patient and the drape, the drape including an opening,
the pressure adapter is mounted above the opening of the drape and includes fittings that allow negative pressure to be applied to the wound through the pressure adapter and the opening; and
(b) a temperature control operatively connected to the dressing, the temperature control including a thermal element that is closely associated with the cover layer and adapted to be placed within the wound.

2. The temperature control vacuum assisted therapy device of claim 1 further comprising at least one secondary pad between the encapsulated pad and the drape.

3. The temperature control vacuum assisted therapy device of claim 1, wherein the thermal element is conformable and includes a coil in the form of a manifold that is capable of containing countercurrent circulating liquid, the coil including a liquid inlet channel and a liquid outlet channel.

4. The temperature control vacuum assisted therapy device of claim 3 further comprising a regulator for regulating the temperature of the liquid in the manifold.

5. The temperature control vacuum assisted therapy device of claim 4, wherein the regulator heats the liquid.

6. The temperature control vacuum assisted therapy device of claim 4, wherein the regulator cools the liquid.

7. The temperature control vacuum assisted therapy device of claim 3 further comprising a pump that pumps the circulating liquid.

8. The temperature control vacuum assisted therapy device of claim 1, wherein the thermal element is conformable and is adhered to the cover layer.

9. The temperature control vacuum assisted therapy device of claim 1, wherein the thermal element is conformable and is integral to the cover layer.

10. The temperature control vacuum assisted therapy device of claim 1, wherein the thermal element is conformable and is fixed to the cover layer.

11. The temperature control vacuum assisted therapy device of claim 1 further comprising a pump that provides negative pressure operably connected to the fittings.

12. The temperature control vacuum assisted therapy device of claim 1, wherein the wound is an open abdomen and the thermal element that is closely associated with the cover layer is adapted to allow thermal transfer between the contents of the bowel and the thermal element.

13. A method of preventing or treating hypothermia in a post-operative abdominal surgery patient that has an open abdomen, the method comprising:
(a) providing a device that includes (i) a dressing having a cover layer, an encapsulated pad, a drape, and a pressure adapter, wherein the cover layer is fenestrated and adapted to be placed within a wound, the encapsulated pad protrudes at least partially through the fenestration of the cover layer and is adapted to allow negative pressure to be applied to the wound, the drape covers the cover layer and encapsulated pad and is capable of providing a seal between the patient and the drape, the drape including an opening, the pressure adapter is mounted above the opening of the drape and including fittings that allow negative pressure to be applied to the wound through the pressure adapter; and (ii) a temperature control operatively connected to the dressing, the temperature control including a thermal element that is closely associated with the cover layer and adapted to be placed within the open abdomen;
(b) applying the device to the open abdomen with the thermal element within the open abdomen;
(c) providing heat to the patient through the thermal element.

14. The method of claim 13 further comprising wrapping the cover layer and the thermal element closely associate with the cover layer over the contents of the bowel.

15. The method of claim 14, wherein the thermal element is conformable.

16. The method of claim 13, wherein the thermal element is conformable and includes a coil in the form of a manifold that is capable of containing countercurrent circulating liquid, the coil including a liquid inlet channel and a liquid outlet channel, the method further comprising circulating warming liquid through the coil.

17. The method of claim 16, wherein the device further includes a regulator for regulating the temperature of the liquid in the manifold, the method further comprising adjusting the temperature in the liquid in the manifold with the regulator.

18. The method of claim 13, wherein the thermal element is conformable and is adhered to the cover layer, the method further comprising wrapping the cover layer and the thermal element closely associate with the cover layer over the contents of the bowel.

19. The method of claim 13, wherein the thermal element is conformable and is integral to the cover layer, the method further comprising wrapping the cover layer and the thermal element closely associate with the cover layer over the contents of the bowel.

20. The method of claim 13, wherein the thermal element is conformable and is fixed to the cover layer, the method further comprising wrapping the cover layer and the thermal element closely associate with the cover layer over the contents of the bowel.

21. A temperature control vacuum assisted wound dressing comprising:
a fenestrated cover layer adapted to be placed within a wound,
an encapsulated pad that protrudes at least partially through the fenestrated cover layer and permits negative pressure to be applied to the wound,
a cover drape, sized to cover the cover layer and encapsulated pad and provide a seal with the patient and define an opening,
a pressure adapter that mounts above the cover drape opening and enables negative pressure to be applied to the wound; and,
a temperature controlled thermal element that is placed within the wound and associated with the cover layer.
